(19) United States
(12) Patent Application Publication
(10) Pub. No.: US 2003/0084500 A1
(43) Pub. Date: May 8, 2003

(54) Diving and swimming mask

(76) Inventors: Fabio Testa, Busalla (IT); Renzo Maggi, Rivanazzano (IT)

Correspondence Address: James C. Wray, Suite 300, 1493 Chain Bridge Road, McLean, VA, 22101, US

(21) Appl. No.: 10/265,903

(22) Filed: Oct. 8, 2002

(30) Foreign Application Priority Data
Oct. 8, 2001 (IT) SV2001A000036

(51) Int. Cl. A61F 9/02

(52) U.S. Cl. 2/431

(57) ABSTRACT

The invention relates to a diving and swimming mask, comprising a mask body (1) which has at least one front opening, and means (3, 4) for firmly securing and/or holding the peripheral edge of at least one transparent member (2) in a water tight manner all along the peripheral edge of said front opening. According to the invention, the mask has at least one, two, three or more members (6) for finishing the peripheral edge of the assembly consisting of the transparent member (2), the mask body (1) and the securing members (3, 4), which members (6) may be added and/or secured at least or only by interlock and/or shape fitting, and after the addition of the means for water tightly securing the transparent member (2) to the mask body (1). In accordance with a preferred embodiment, the mask has a buckle 7, at each lateral edge, for engaging an end of a strap, the buckles being held by a buckle holding member (6), and each buckle holding member (6) consisting of at least one corresponding finishing member as described above.

The invention relates to a diving and swimming mask, comprising a mask body, made of a soft material, which has at least one front opening all around the eyes of the user, and means for firmly securing and/or holding the peripheral edge of at least one transparent member, particularly a lens, in a water tight manner all along the peripheral edge of said front opening.

Many types of diving masks as described hereinbefore are known, each being differentiated by aesthetic peculiarities depending on fashion and customer taste issues and/or on technical complexity, which is related to the more or less professional use for which the mask is designed. Typically, each mask type is manufactured separately from other mask types, in relation to the desired aesthetic and/or technical characteristics. Aesthetic characteristics are mainly determined by the conformation of the means for water tightly securing the transparent member to the mask body. Hence, each mask type with a different color or aspect requires a separate or dedicated manufacturing process. This adversely affects the manufacturing cost of masks and the price whereat they are sold to end users, due to the need of implementing a separate manufacturing line for each mask type.

Therefore, this invention has the object of obviating, by very simple and inexpensive means, the above adverse situation, thereby simplifying the manufacturing process for the various mask types and reducing their manufacturing costs. Particularly, as better explained below, the object of the invention is to allow the fabrication of a large portion of the mask on a single line, regardless of the desired final characteristics thereof, and to complete the addition of aesthetic and/or technical characterization members, which might differentiate one mask from the other, in a subsequent step. Another object of this invention is to improve certain mask parts to improve user comfort and user-friendliness.

The invention achieves the above purposes by providing a mask as described hereinbefore, which has at least one, two, three or more members for finishing the peripheral edge of the assembly consisting of the transparent member, the mask body and the members for securing said transparent member to the mask body, which members may be applied and/or fastened at least or only by interlock and/or form fitting.

These finishing members may consist of frame members, to be arranged in separate and spaced positions along the periphery of the assembly consisting of the transparent member, the mask body and the securing members, or may consist of an open or closed, continuous annular member.

These finishing members may be removably secured by means of interlocking and/or shape fitting and/or mechanical fastening means. As an alternative, they may be secured in a non removable manner, by welding and/or gluing and/or by any other chemical/physical bond arrangement, which may or may not provided in combination with mechanical fastening means.

In accordance with a preferred embodiment, each finishing member may consist of an open profile, whose shape is substantially complementary to the cross section of the assembly composed of the transparent member, the mask body and the means for water tightly securing the latter to each other, in the engagement section.

Each finishing member may have snap lock means, cooperating with complementary snap lock means on the assembly composed of the transparent member, the mask body and the means for water tightly securing the latter to each other. In accordance with a preferred arrangement, these means may be provided on the means for water tightly securing the transparent member to the mask body.

Advantageously, the finishing member/s may be added after the addition of the means for water tightly securing the transparent member against the mask body.

The above description apparently shows a first basic advantage of this invention, that is the possibility, better explained below, of separating the steps of assembling the mask body and the transparent member and those of finishing and aesthetically and/or technically characterizing the mask. In other words, the guiding principle of this invention allows to manufacture the finished portion of the mask, i.e. composed of a body, a transparent member and means for water tightly securing said transparent member to the body, in a single type or a limited number of types, and to perform the process of adding aesthetic and/or technical finishing members, as mentioned above, which might characterize and distinguish even importantly one mask type from another, in a subsequent step.

Like prior art masks, the inventive mask may have a buckle, at one but preferably at both lateral edges, which is designed to engage an end of a strap for holding the mask to the user's face. At least one, but preferably both buckles may be held by a buckle-holding member, on each lateral edge of the assembly composed of the mask body, the transparent member and the means for securing the latter. According to a very important characteristic of the invention, the buckle-holding member/s may be advantageously associated to or formed by at least one corresponding finishing member which has, therefore, both aesthetic and technically functional purposes. Thanks to this arrangement, the above considerations may be extended to the possibility of differentiating the technical complexity of the mask, or simply of customizing the means for engaging the fastening strap, both functionally and aesthetically, always starting from a single type of finished portion of the mask or from a limited number of such types.

Each buckle-holding member may consist of a rear buckle holding extension, with reference to the observation direction of the mask user, which starts from an engagement member on the lateral edge of the means for securing the transparent member to the mask body.

Said engagement member may have a recess whose cross section substantially corresponds or complements that of the means for securing the transparent member to the mask body, in the engagement area, in much the same manner as described for simple finishing means. In order to obtain a more effective holding action, the latter may lay over at least three sides or through an angle above 90°, preferably of substantially 180° or above, of said means for securing the transparent member to the mask body.

Each engagement member may have such a shape and longitudinal extension as to lay over both the lateral edge and the upper and lower curved areas, whereat the lateral edge is linked to the upper and lower edge of the means for securing the transparent member to the mask body. This arrangement allows the engagement member to be more effectively secured to the edge of the mask and provides an enhanced aesthetic characterization, as well as a higher mechanical strength of the engagement member itself.

The two engagement members at the two opposite lateral edges of the mask may extend along a small portion of the upper and/or lower edges of the means for securing the transparent member to the mask body, or may extend along a greater portion and be in mutual end contact and/or partial end overlapping position, with or without mutual engagement, along said upper and/or lower edges.

Each engagement member may be made of a relatively rigid material, preferably plastic, and be secured to the means for securing the transparent member to the mask body by mechanical snap fitting and/or by physical/chemical bond, such as gluing, welding, or the like.

Advantageously, in the engagement portion of the edge of means for securing the transparent member to the mask body and along the wall of the engagement member, which lays over said edge, there may be provided one or more complementary projections or teeth and one or more recesses or apertures for snap fitting connection. Particularly, said snap fitting connection may be arranged to be of the removable type, for instance in order to allow aesthetic and/or technical customization of this or other parts provided with the mask by the customer.

Particularly, a preferably elongated, backwardly extending interlocking tooth may be provided in a substantially central position of each lateral edge of the means for securing the transparent member. A corresponding elongated aperture may be provided along the corresponding wall of the engagement member, for engagement of the tooth, to prevent the engagement member from spreading apart.

As mentioned above, the engagement members and the associated rear buckle-carrying extensions may advantageously act both as aesthetic conformation members of the mask and as technical characterization members. To this end, these members may be made of one of more materials and/or colors equal to or different from each other and other mask parts.

The rear extension may form a compartment for reversal or return of the strap around the buckle. The latter may include strap locking means which may be stationary or operated to release the strap in the loosening and/or fastening direction. As described in greater detail below in the description of the drawings, in this second arrangement, the buckle function is also performed by the rear extension or a removable part thereof.

In accordance with a first simpler arrangement, the buckle locking means, which cooperate with the strap, may be mounted directly on the rear extension of the buckle-holding member.

Alternatively, the buckle may be a separate member to be joined to the rear extension.

To this end, the buckle locking means, cooperating with the strap, may be fitted in a box-like member which forms the body of the separate buckle.

Said buckle and/or box thereof may have means for mutual snap fit engagement with said rear extension.

As a whole, the above arrangements may be provided in any mask type, provided it is appropriately arranged therefor.

A particularly simple and inexpensive preferred embodiment provides that the means for securing the transparent member to the mask body may consist of two mounts, a front mount and a rear mount, the peripheral edge of the front opening of the mask body and the peripheral edge of the transparent member being water tightly clamped therebetween. In an even simpler embodiment, the front mount may be replaced by means for direct water tight bond with the transparent member, such as silicone or the like.

The rear mount may have a preferably continuous, inner peripheral flange, whose front face is in contact with the rear face of an outer peripheral flange which is provided along the edge of the front opening of the mask body, the peripheral edge of the transparent member resting on said second flange.

According to an improvement designed to improve the water tightness of the assembly, the inner peripheral flange of the rear mount may have a preferably continuous first throat or groove, whereas the outer peripheral flange of the mask body may have a preferably continuous tongue, which is complementary in shape to the first throat and has a backward orientation, for engagement in said first throat, whereas the flange side whereon the transparent member rests may be flattened.

To the same purpose, the rear mount may have a second, preferably continuous peripheral throat, which extends radially outwards with respect to the flange having the first throat, whereas the front mount may consist of a preferably continuous backwardly extending tongue, which is complementary in shape to the second throat and is engaged therein, and of a preferably continuous inner peripheral flange, which starts from the front edge of said tongue and lays over the peripheral edge of the transparent member and the underlying flange of the mask body to clamp said transparent member against the mask body and against the rear mount.

The two mounts may be joined together by mechanical connection and/or by a chemical and/or physical bond, particularly by ultrasonic welding, and may form, with the transparent member and the mask body, a finished mask part, whereon the members that hold the strap fastening buckles may be added later.

In accordance with a preferred embodiment, a preferably elongated, backwardly extending interlocking tooth may be provided in a substantially central position of each lateral edge of the rear mount. Along the corresponding wall of the engagement member, which carries the rear extension for housing the buckle or the removable buckle body, a corresponding elongated aperture may be provided for engagement of the tooth, to prevent the engagement member from spreading apart.

According to an improvement, the front of said tooth, which is turned toward the outside of the mask may have an inclined lead-in end surface, for an easier insertion in the aperture, whereas the inner front is oriented transverse to the direction in which the engagement member is disengaged from the aperture, and may itself have a certain inclination, which allows disengagement of the engagement member from the assembly of the two joined mounts, with a predetermined disengagement effort.

According to an additional improvement, to be detailed in the description of the drawings, the outer peripheral edge of the assembly formed by the two joined mounts may have a continuous radial widened portion, having the form of a central step which starts from two continuous side bands, whereas the engagement member which carries the rear extension may have a corresponding extension, to generate strengthening folds for an optimized shape-fit and a better mechanical retaining action. It shall be noted that the two joined mounts as described above form a basic, minimized frame, which is only formed by the substantially important parts for water tightly securing the transparent member against the mask body. Therefore, the guiding principle of this invention may also apply to masks whose mounts are very different from those described above, particularly larger.

The advantages of this invention are self-evident from the above disclosure and consist in that the mask core may be manufactured separately from the members for finishing and/or supporting the strap tensioning and locking means. Hence, these members may be fabricated according to different aesthetic and/or functional characteristics and/or quality levels. According to the customer-selected aesthetic aspect and to the associated costs, several different additional members may be manufactured, all having the same inner shape but allowing the provision of any external form. In practice, it is possible to fabricate a wide range of mask types, even though a single mask body type, or a small number of types are only kept in stock. In other words, a wide range of masks may be provided, all having different aesthetic characteristics and/or different qualities of the strap securing members, while keeping a single type of assembly of the transparent member and mask body, regardless of the aesthetic characteristics and of the technical quality of the finished mask.

Further characteristics and improvements will form the subject of the dependent claims.

The characteristics of the invention and the advantages derived therefrom will be more apparent from the following detailed description of the annexed drawings, in which:

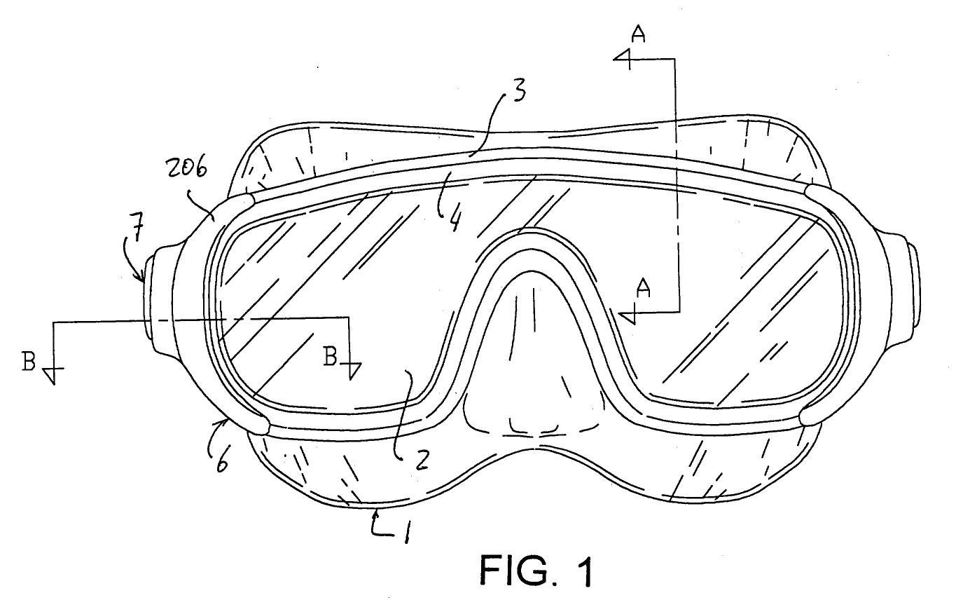

FIG. 1 is a perspective view of a first embodiment of a mask according to the invention.

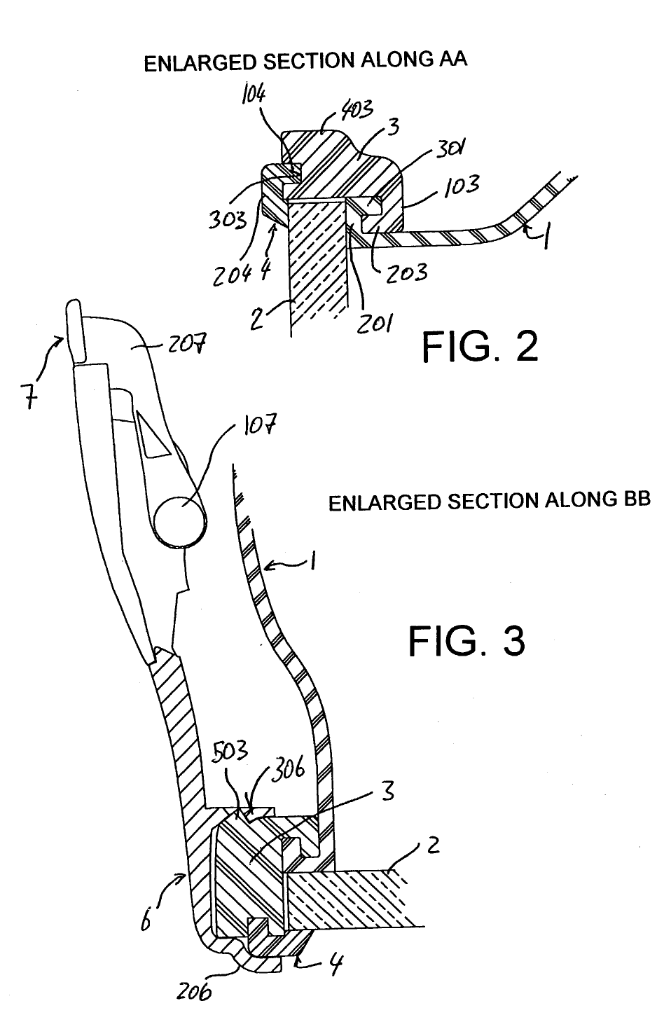

FIG. 2 is a sectional view of the mask as seen through the line AA of FIG. 1.

FIG. 3 is a sectional view of the mask as seen through the line BB of FIG. 1.

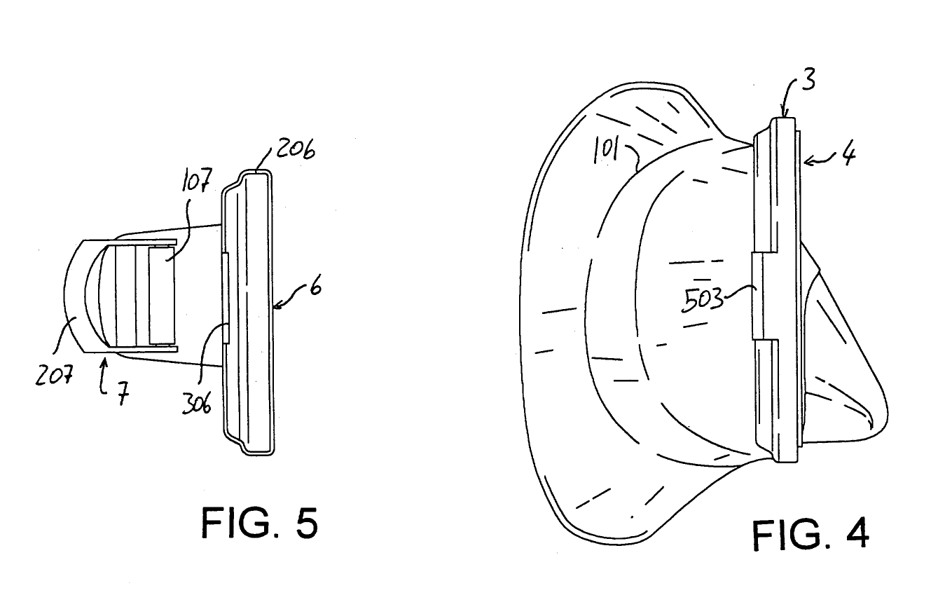

FIG. 4 is a side view of the mask, wherein the removable buckle supporting means are omitted.

FIG. 5 is a front view, as seen from the inside, of the removable buckle supporting means.

The figures show a preferred embodiment of the mask according to this invention, which has a one-lens arrangement and an anatomic nose part, to facilitate compensation breathing operations. Anyway, the teaching of this invention may also advantageously apply to different mask types, e.g. binocular masks.

The mask has a mask body 1 made of a soft and comfortable material, for instance rubber or the like. This body 1 has a soft rear flange for water tight adhesion of the mask body 1 to the user face. The mask further includes a front transparent member, typically a lens 2, which is secured in a water tight manner to the peripheral edge of a front opening of the mask body 1 around the user's eyes. FIG. 2 shows in detail the lens securing area, with two mounts, a rear mount 3 and a front mount 4 that water tightly clamp the mask body 1 and the lens 2 therebetween. The two mounts 3, 4 are made of a relatively rigid material, particularly plastic, and have the shape of continuous and closed frames which surround the whole peripheral edge of the lens 2 and interlock to provide the desired water tight clamping effect. To this end, the whole peripheral edge of the front opening of the mask body 1 has an external U-shaped frame element, which generates a continuous groove whose concave part is oriented rearwards. This frame element is preferably made of the same soft material as the mask body 1 to assist water tightness and consists of a continuous peripheral flange 201, which extends radially outwards from the edge of the front aperture and of a continuous tongue 301 which extends rearwards from the outer peripheral edge of the flange 201. By this arrangement, the frame element of the body 1 has a groove on its rear face, whereas the front face is flattened and in contact with the lens 2.

The rear mount 3 has an inner, U-shaped frame member, whose concave part is oriented to the front. This frame element consists of a continuous flange 103 which extends radially inwards and of a tongue which extends at the front from the inner peripheral edge of the flange 103. Thanks to this arrangement, the frame element of the rear mount 3 has a groove on its front face, wherein the tongue 301 of the mask body interlocks, whereas the tongue 302 engages in the groove of the frame member of the mask body 1.

The rear mount 3 further has a second continuous throat or peripheral groove 303, which extends radially outwards with respect to the groove formed by the flange 103 and the tongue 203, whereas the front mount 4 has an additional tongue 104 which extends backwards and whose shape is complementary to the second groove 303. In the assembled condition, the tongue 104 is engaged in the groove 303 and the front mount 4 presses the lens 2 against the front face of the flattened edge of the front opening of the mask body thanks to the fact that the front mount extends radially inwards for a certain portion of the peripheral edge of the lens 2, thereby forming, as a whole, an L-shaped frame. Then, the two mounts may be joined together and to the mask body 1, preferably by ultrasonic welding.

The mask obtained thereby is a basic element, that can be diversified by adding aesthetic finishing and/or functional members. In the embodiment as shown in the figures, the mask has a pair of members 6, each having a rear extension 7 which holds a rocking support member 107 that is designed to lock a strap, not shown. The rocking support member has a strap releasing or disengaging pushbutton 207, which is used to loosen the strap.

FIGS. 3 and 4 show one of these members 6, which consists of a rear extension 7 for housing the rocking support member 107, which extension is provided in the form of an inwardly open shell, starting from an engagement member 206 on a lateral edge of the assembly of the two joined mounts 3, 4. This engagement member 206 is formed by an additional frame segment whose longitudinal edges overlap at the front and at the back of the two joined mounts 3, 4 in the area of the lateral edge and for a limited portion of the upper and lower edges. Hence, the engagement member 206 has an inner groove, whose cross section is substantially complementary to that of the two mounts 3, 4, that are joined in the engagement area. According to an advantageous improvement, the outer peripheral edge of the assembly of the two joined mounts 3, 4 has a central continuous step 403, which extends radially outwards, whereas the engagement member 206 has a corresponding internal shape, so that it is provided with longitudinal strengthening folds for further assisting shape fit.

An interlocking tooth 503 is provided in a substantially central position of each lateral edge of the rear mount 3, which tooth is elongated in the vertical direction with reference to a mask worn by a standing user. This tooth 503 extends backwards, whereas a corresponding elongated aperture 306 is provided along the corresponding wall of the inner recess of the engagement member 206, for engagement of the tooth 503 to prevent the engagement member 206 from spreading apart and to prevent the member 206 from sliding on the two joined mounts 3, 4. FIG. 3 clearly shows the lead-in surface 603 of the tooth 305 for insertion in the aperture 306. By this arrangement, the buckle holding member 6 may be mechanically snap fitted on the lateral edge of the mask and be possibly replaced by the user, e.g. with a member having a different color and/or different technical characteristics for mask customization.

The rear extension 7 forms a compartment for reversal and return of the strap (not shown), around the rocking support member, which is of such type as to allow operation thereof to release the strap toward loosening and/or fastening it by finger pressure on the end of an operating button 207. As is known, this type of buckle consists of a rocking frame 107 which only rocks about a substantially vertical axis, and of a stationary abutment part which, in the illustrated embodiment, is formed by the inner wall of the rear extension 7 of the buckle holding member 6. This rear extension 7 is substantially U-shaped, and the rocker frame 107 is pivoted at holes provided on the opposite inner faces of the U arms. In this case, the rear extension 7 may be considered as a part of the buckle, which may also be of a different type, e.g. of the stationary type. The buckle as shown in the figures provides the advantage that the elastic means for holding the strap in a locked position in which it is engaged with the buckle, particularly with the rocking frame 107, consist of the strap itself, i.e of the fastening or loosening action exerted thereon, without having to use any other means, such as springs or the like.

The rocking frame 107 may be also accommodated in a box, forming a part of the buckle, which box has snap interlocking means in an appropriate seat formed by the rear extension of the member 6. These interlocking means may be made in any manner and possibly provided with quick release means to be easily accessed from the outside.

Obviously, the invention is not limited to the embodiment described and illustrated herein, but may be greatly varied, without departure from the guiding principle disclosed above and claimed below.

1. A diving and swimming mask, comprising a mask body (1), made of a soft material, which has at least one front opening all around the eyes of the user, and means (3, 4) for firmly securing and/or holding the peripheral edge of at least one transparent member (2), particularly a lens (2), in a water tight manner all along the peripheral edge of said front opening, characterized in that it has as at least one, two, three or more members (6) for finishing the peripheral edge of the assembly consisting of the transparent member (2), the mask body (1) and the securing members (3, 4), which members (6) may be added and/or secured at least or only by interlock and/or shape fitting.

2. A mask as claimed in claim 1, characterized in that said finishing members are frame members (206), arranged in separate and spaced positions along the periphery of the assembly consisting of the transparent member (2), the mask body (1) and the securing members (3, 4), or an open or closed, continuous annular member.

3. A mask as claimed in claim 1 or 2, characterized in that the finishing members (6) are removably secured by means of interlocking (503, 306) and/or shape fitting and/or mechanical fastening means.

4. A mask as claimed in one or more of the preceding claims, characterized in that each finishing member (6) consists of an open profile (206), whose shape is substantially complementary to the cross section of the assembly composed of the transparent member (2), the mask body (1) and the means (3, 4) for water tightly securing the latter to each other, in the engagement section.

5. A mask as claimed in one or more of the preceding claims, characterized in that each finishing member (6) has snap lock means (306), cooperating with complementary snap lock means (503) on the assembly composed of the transparent member (2), the mask body (1) and the means (3, 4) for water tightly securing the latter to each other, which snap lock means are preferably provided on said means (3, 4) for water tightly securing the transparent member (2) to the mask body (1).

6. A mask as claimed in one or more of the preceding claims, characterized in that the finishing members (6) are secured in a non removable manner, by further welding and/or gluing and/or by any other chemical/physical bond arrangement.

7. A mask as claimed in one or more of the preceding claims, characterized in that the finishing member/s (6) may be added after the addition of the means (3, 4) for water tightly securing the transparent member (2) to the mask body.

8. A mask as claimed in one or more of the preceding claims, characterized in that it has a buckle, at each lateral edge, for engaging an end of a strap (5), at least one, but preferably both buckles being held by a buckle holding member (6), and the buckle holding member/s consisting of at least one corresponding finishing member (6).

9. A mask as claimed in one or more of the preceding claims, characterized in that each buckle-holding member (6) consists of a rear buckle holding extension (7), with reference to the observation direction of the mask user, which starts from an engagement member (206) on the lateral edge of the means (3, 4) for securing the transparent member (2) to the mask body (1).

10. A mask as claimed in one or more of the preceding claims, characterized in that said engagement member (206) has a recess whose cross section substantially corresponds or complements that of the means (3, 4) for securing the transparent member (2) to the mask body (1), in the engagement area, and which lays over at least three sides or through an angle above 90°, preferably of substantially 180° or above, of said means (3, 4) for securing the transparent member (2) to the mask body (1).

11. A mask as claimed in one or more of the preceding claims, characterized in that each engagement member (206) has such a shape and longitudinal extension as to lay over both the lateral edge and the upper and lower curved areas, whereat the lateral edge is linked to the upper and lower edges of the means (3, 4) for securing the transparent member (2) to the mask body (1).

12. A mask as claimed in one or more of the preceding claims, characterized in that the two engagement members (206) extend along a small portion of the upper and/or lower edges of the means (3, 4) for securing the transparent member (2) to the mask body (1), or may extend along a greater portion and be in mutual end contact and/or partial end overlapping position, with or without mutual engagement, along said upper and/or lower edges.

13. A mask as claimed in one or more of the preceding claims, characterized in that each engagement member (206) is made of a relatively rigid material, preferably plastic, and is secured to the means (3, 4) for securing the transparent member (2) to the mask body (1) by mechanical snap fitting and/or by physical/chemical bond.

14. A mask as claimed in one or more of the preceding claims, characterized in that, in the engagement portion of the edge of means (3, 4) for securing the transparent member (2) to the mask body (1) and along the wall of the engagement member (206), which lays over said edge, there may be provided one or more complementary projections or teeth (503) and one or more recesses or apertures (306) for snap fitting connection.

15. A mask as claimed in one or more of the preceding claims, characterized in that a preferably elongated, backwardly extending interlocking tooth (503) is provided in a substantially central position of each lateral edge of the means (3, 4) for securing the transparent member (2) to the mask body (1), whereas a corresponding elongated aperture (306) is provided along the corresponding wall of the engagement member (206), for engagement of the tooth (503) to prevent said engagement member (206) from spreading apart.

16. A mask as claimed in one or more of the preceding claims, characterized in that the engagement members (206) and the associated rear buckle-holding extensions are aesthetic conformation and/or technical characterization members for the mask.

17. A mask as claimed in one or more of the preceding claims, characterized in that said rear extension forms a housing for reversal or return of the strap around the buckle, which includes strap locking means (107), that may be stationary or operated to release the strap toward loosening and/or fastening it.

18. A mask as claimed in one or more of the preceding claims, characterized in that the buckle locking means (107), which cooperate with the strap, are mounted directly on said rear extension (7) of the buckle-holding member (6).

19. A mask as claimed in one or more of the preceding claims, characterized in that the buckle is a separate element, to be added to the rear extension (7), in a permanent or removable manner.

20. A mask as claimed in one or more of the preceding claims, characterized in that the buckle locking means (107), which cooperate with the strap, are mounted in a box-like element which forms the separate buckle body.

21. A mask as claimed in one or more of the preceding claims, characterized in that said buckle and/or said box thereof have means for mutual snap fit engagement with said rear extension (7).

22. A mask as claimed in one or more of the preceding claims, characterized in that said means for mutual snap fit engagement have means for mutual release of the buckle box from the rear housing extension (7).

23. A mask as claimed in one or more of the preceding claims, characterized in that the means for securing the transparent member (2) to the mask body (1) consist of two mounts, a front mount (4) and a rear mount (3), the peripheral edge of the front opening of the mask body (1) and the peripheral edge of the transparent member (2) being water tightly clamped therebetween.

24. A mask as claimed in one or more of the preceding claims, characterized in that the rear mount (3) has a preferably continuous, inner peripheral flange (103, 203), whose front face is in contact with the rear face of an outer peripheral flange (201) which is provided along the edge of the front opening of the mask body (1), the peripheral edge of the transparent member (2) resting on said second flange (201, 301).

25. A mask as claimed in one or more of the preceding claims, characterized in that the inner peripheral flange (103, 203) of the rear mount (3) has a preferably continuous first throat or groove, whereas the outer peripheral flange (201) of the mask body (1) has a preferably continuous tongue, which is complementary in shape to the first throat and has a backward orientation, for engagement in said first throat, whereas the flange side (201) whereon the transparent member (2) rests may be flattened.

26. A mask as claimed in one or more of the preceding claims, characterized in that the rear mount (3) has a second, preferably continuous peripheral throat (303), which extends radially outwards with respect to the flange (103, 203) having the first throat, whereas the front mount (4) consists of a preferably continuous backwardly extending tongue (104), which is complementary in shape to the second throat (303) and is engaged in said second throat (303), and of a preferably continuous inner peripheral flange, which starts from the front edge of said tongue (104) and lays over the peripheral edge of the transparent member (2) and the underlying flange (201) of the mask body (1) to clamp said transparent member (2) against the mask body (1) and against the rear mount (3).

27. A mask as claimed in one or more of the preceding claims, characterized in that the two mounts (3, 4) may be joined together by a mechanical connection and/or by a chemical and/or physical bond, particularly by ultrasonic welding, and form, with the transparent member (2) and the mask body (1), a finished mask part, whereon the members (6) that hold the strap fastening buckles are added later, said members being fabricated as claimed in one or more of claims 1 to 22.

28. A mask as claimed in one or more of the preceding claims, characterized in that a preferably elongated, backwardly extending interlocking tooth (503) is provided in a substantially central position of each lateral edge of the rear mount (3), whereas a corresponding elongated aperture (306) is provided along the corresponding wall of the engagement member which holds the rear extension for housing the buckle or the removable body thereof (206), for engagement of the tooth (503) to prevent said engagement member (206) from spreading apart.

29. A mask as claimed in one or more of the preceding claims, characterized in that the front of said tooth (503), which is turned toward the outside of the mask has an inclined lead-in end surface (603), for an easier insertion in the aperture (306), whereas the inner front is oriented transverse to the direction in which the engagement member (206) is disengaged from the aperture (306), and may itself have a certain inclination, which allows disengagement of the engagement member (206) from the assembly of the two joined mounts (3, 4), with a predetermined disengagement effort.

30. A mask as claimed in one or more of the preceding claims, characterized in that the outer peripheral edge of the assembly formed by the two joined mounts (3, 4) has a continuous radial widened portion, having the form of a central step (403) which starts from two continuous side bands, whereas the engagement member (206) which holds the rear extension has a corresponding extension, to generate strengthening folds for an optimized shape-fit and a better mechanical retaining action.

31. A swimming mask, wholly or partly as described, illustrated and for the purposes stated herein.
